(19) United States
(12) Patent Application Publication
(10) Pub. No.: US 2021/0007995 A1
(43) Pub. Date: Jan. 14, 2021

(54) POLYMER MIXTURE WITH RESISTANCE AGAINST THE INFLUENCE OF ETHANOL

(71) Applicant: Evonik Operations GmbH, Essen (DE)

(72) Inventors: Thomas Endres, Hoover, AL (US); Christian Meier, Darmstadt (DE); Florian Hermes, Haltern am See (DE); Jessica del Rosario Ferrand, Eppertshausen (DE); Herbert Jung, Karlstein (DE); Thomas Eurich, Hanau (DE); Jan Hendrik Schattka, Darmstadt (DE)

(73) Assignee: Evonik Operations GmbH, Essen (DE)

(21) Appl. No.: 15/733,592

(22) PCT Filed: Feb. 27, 2019
(86) PCT No.: PCT/EP2019/054817

§ 371 Date: Sep. 8, 2020

(30) Foreign Application Priority Data
Mar. 9, 2018 (EP) 18160836.5

(51) Int. Cl. A61K 9/50; C09D 133/10; C08F 220/18

(52) U.S. Cl. CPC A61K 9/5026; C08F 220/1808; A61K 9/5089; C09D 133/10

(57) ABSTRACT

A polymer mixture includes 10 to 90 wt. % of polymer 1 and 10 to 90 wt. % of polymer 2, the total being 100 wt. %. Polymer 1 is polymerized from (a1) 70-95 wt. % of C₁-C₁₂ alkylester of (meth)acrylic acid, and (b1) 5-30% wt. % of C₂-C₈ alkyl ester of (meth)acrylic acid with a quaternary cationic group in the alkyl group. Polymer 2 is polymerized from (a2) 70-95 wt. % of C₁-C₁₂ alkylester of (meth)acrylic acid, and (c2) 5-30 wt. % of C₂-C₆ hydroxy-alkylester of (meth)acrylic acid. Monomers (a1) and (b1) add up to 98-100 wt. %, and most preferred to 100 wt. %. Monomers (a2) and (c2) add up to 98-100 wt. %, most preferred to 100 wt %.

### BACKGROUND

Alcohol resistant enteric formulations are known from
WO 2009/036811: PH-dependent controlled release pharmaceutical composition for non-opioids with resistance against the influence of ethanol.
WO 2009/036812: PH-dependent controlled release pharmaceutical opioid composition with resistance against the influence of ethanol.
WO 2010/034342: PH-dependent controlled release pharmaceutical opioid composition with resistance against the influence of ethanol.
WO 2010/034344: PH-dependent controlled release pharmaceutical composition for non-opioids with resistance against the influence of ethanol.
WO 2012/171884: Gastric resistant pharmaceutical or nutraceutical composition with resistance against the influence of ethanol.
A. Krieg, E. Arici, et al. (2014). “Toward pH-Responsive Coating Materials—High-Throughput Study of (Meth)acrylic Copolymers.” ACS Combinatorial Science 16(8): 386-392.
WO 2010/105672: Controlled release pharmaceutical composition with resistance against the influence of ethanol employing a coating comprising neutral vinyl polymers and excipients.
WO 2010/105673: Controlled release pharmaceutical composition with resistance against the influence of ethanol employing a coating comprising a polymer mixture and excipients.
WO 2015/121189: Pharmaceutical or nutraceutical composition with sustained release characteristic and with resistance against the influence of ethanol.
WO 2014/151797: Extended release formulations resistant to alcohol dose dumping.
WO 2016/193034: Pharmaceutical or nutraceutical composition with resistance against the influence of ethanol.
Y. Rosiaux, C. Velghe, et al. (2013). “Ethanol-resistant ethylcellulose/guar gum coatings—Importance of formulation parameters.” European Journal of Pharmaceutics and Biopharmaceutics 85(3, Part B): 1250-1258.
Y. Rosiaux, C. Velghe, et al. (2014). “Mechanisms Controlling Theophylline Release from Ethanol-Resistant Coated Pellets.” Pharmaceutical Research 31(3): 731-741.
C. Y. Gujjar, B. C. Rallabandi, et al. (2015). “Development and Optimization of a Novel Prolonged Release Formulation to Resist Alcohol-Induced Dose Dumping.” AAPS PharmSciTech: 1-8.
WO 2012/171884A1 describes gastric resistant pharmaceutical or nutraceutical composition with resistance against the influence of ethanol. Disclosed is a (meth)acrylate copolymer comprising polymerized units of 10 to 40% by weight of acrylic or methacrylic acid, 10 to 80% by weight of a C₄- to C₁₈-alkyl ester of acrylic or methacrylic acid and optionally 0 to 60% by weight of another vinylic monomer, whereby the release of the pharmaceutical or nutraceutical active ingredient is not more than 10% under in-vitro conditions at pH 1.2 after 2 hours in medium according to USP with and without the addition of 20% (w/w) ethanol.
U.S. Pat. No. 4,737,357 describes a method for producing a film-forming aqueous dispersions and coating agent for pharmaceuticals comprising a (meth)acrylate copolymer which is composed of free-radical polymerized methyl methacrylate, ethylacrylate, and 2-Trimethylammonium-ethyl-methacrylate-chloride.
DE102005024614A1 describes the use of polymer mixtures for the production of coated pharmaceutical formulations and pharmaceutical formulations with a mixed polymer coating. The mixed polymer coating comprises a mixture of a polymer (I) and a polymer (II). Polymer (I) comprises 90 to 100% by weight of polymerized units of 40 to 95% by weight of C1- to C4-alkyl esters of acrylic or methacrylic acid and 5 to 60% by weight of (meth)acrylate monomers having an anionic group and 0 to 10% by weight of further vinylically polymerizable monomers.

### General Definitions

Singular forms like “a”, “an”, “the” or “another” as used in the description or in the claims shall be understood as to include the plural of the defined subject within the given definition or limits as well if not stated explicitly otherwise.

Singular terms like “a C₁-C₁₂ alkylester of acrylic acid or of methacrylic acid” shall be understood as “C₁-C₁₂ alkylester of acrylic acid or C₁-C₁₂ alkylester of methacrylic acid” and shall also include one or more of these monomers and any mixtures thereof.

The terms “comprises” or “is comprising” shall be understood as including the terms “essentially comprises” or “is essentially comprising” and “consists” or “consisting of”.

A “C₂-C₈ alkyl ester of acrylic acid or of methacrylic acid with a quaternary cationic group in the alkyl group” means that the alkyl group is substituted with a quaternary cationic group, preferably with a quaternary ammonium group. Examples for these well-known monomers are 2-Trimethylammonium-ethyl-methacrylate-chloride (TMAEMC) or 2-Trimethylammonium-propyl-methacrylate-chloride (TMAPMC).

### SUMMARY OF THE INVENTION

Pharmaceutical or nutraceutical compositions are designed to release the active ingredient in a manner of reproducible release curves. This shall result in desirable and reliable blood level profiles which shall provide an optimal therapeutic effect. If the blood level concentrations are too low, the active ingredient will not cause a sufficient therapeutic effect. If the blood level concentrations are too high, this may cause toxic effects. In both cases non optimal blood level concentrations of an active ingredient can be dangerous for the patient and shall therefore be avoided. A problem exists in that the ideal ratios assumed for the release of active ingredient during the design of a pharmaceutical or nutraceutical composition can be altered by the general living habits, thoughtlessness or by addictive behaviour of the patients with respect to the use of ethanol or ethanol-containing drinks. In these cases, the pharmaceutical or nutraceutical form which is actually designed for an exclusively aqueous medium is additionally exposed to an ethanol-containing medium of greater or lesser strength. Since health authorities like for instance the US Food and Drug Administration (FDA) focus more and more on the ethanol problem, ethanol resistance may be an important registration requirement in the near future. The problem is severe especially for pharmaceutical compositions but of course also exist for nutraceutical compositions.

Since not all patients or customers are aware of the risk of simultaneous taking of a controlled release pharmaceutical or nutraceutical form and ethanol-containing drinks or do not follow or are not able to follow appropriate warnings, advice or recommendations, there is a demand for controlled release pharmaceutical or nutraceutical compositions, especially for gastric resistant or sustained release pharmaceutical or nutraceutical compositions, such that their mode of action is affected as little as possible by the presence of ethanol.

Conventional extended or sustained release pharmaceutical or nutraceutical compositions if coated or uncoated are usually not resistant to alcohol at all. Therefore, one object of the present application was to provide extended or sustained release pharmaceutical or nutraceutical compositions, which are resistant against the influence of ethanol.

Especially there is a problem for dosage forms with sustained release characteristic. These kinds of formulations are usually coated with water-insoluble polymers or copolymers onto a core comprising a pharmaceutical or nutraceutical active ingredient. The release of the biologically active ingredient, e.g. the pharmaceutical or nutraceutical active ingredient is sustained which means more or less constantly over the time (zero order release) and independent from the pH of the environment. The release of the pharmaceutical or nutraceutical active ingredient under in-vitro conditions after 2 hours at pH 1.2 in simulated gastric fluid according to USP (for instance USP 32) and subsequent change of the medium to buffered medium of pH 6.8 according to USP may for instance be in the range of 2 to 98, 30 to 90, 40 to 80% in a total time, including the 2 hours of the pH 1.2 phase, of 4 to 12, 4 to 8 or 6 to 10 hours.

However the presence of ethanol in concentrations of 5, 10, 20 or 40% (volume/volume) in the gastric fluid usually leads to an increase to the release rates already in the stomach. Thus an effective protection against the influence of ethanol should prevent such an undesired increase of pharmaceutical or nutraceutical active ingredient in the stomach but also in the intestine.

Thus the presence of ethanol in concentrations of 5, 10, 20 or 40% (volume/volume) under in-vitro conditions after 2 hours at pH 1.2 in simulated gastric fluid according to USP (for instance USP 32) shall not severely influence the intended sustained or extended release rates at pH 1.2. Furthermore, the presence of ethanol in concentrations of 5, 10, 20 or 40% (volume/volume) under in-vitro conditions after 2 hours in pH 1.2 medium according to USP (for instance USP 32) and subsequent change of the medium to buffered medium of pH 6.8 according to USP without ethanol, shall not severely influence the intended sustained or extended release rates at pH 1.2 and at pH 6.8.

Polymers like EUDRAGIT® RL, EUDRAGIT® RS or EUDRAGIT® NM are widely used in pharmacy for the coating of sustained release dosage forms. However, these polymers are not resistant against the influence of ethanol. Thus, there is a need for new polymers or polymer mixtures with similar sustained release but also ethanol resistance properties.

The objects are solved as claimed.

### Details of the Invention

Polymer Mixture

The invention is concerned with a

polymer mixture, comprising 10 to 90% by weight of a polymer 1 and 10 to 90% by weight a polymer 2, wherein the weight percentages of polymer 1 and polymer 2 add up to 100%, and

wherein the polymer 1 is a polymer, polymerized from a monomer mixture comprising the monomers

(a1) 70 to 95% by weight of a C₁-C₁₂, C₂-C₁₀ or a C₂-C₈ alkylester of acrylic acid or of methacrylic acid, and

(b1) 5 to 30% by weight of a C₂-C₈ alkyl ester of acrylic acid or of methacrylic acid with a quaternary cationic group, preferably a quaternary ammonium group, in the alkyl group, and wherein the polymer 2 is a polymer polymerized from a monomer mixture comprising the monomers

(a2) 70 to 95% by weight of a C₁-C₁₂, C₂-C₁₀ or a C₂-C₈ alkylester of acrylic acid or of methacrylic acid, and

(c2) 5 to 30% by weight of a C₂-C₆ or a C₂-C₄ hydroxy-alkylester of acrylic acid or methacrylic acid.

The polymer mixture may comprise a ratio by weight of polymer 1 to polymer 2 of from 5:1 to 1:5, preferably from 2:1 to 1:2.

Generally each “% by weight” range for each monomer may be combined with each weight-% range for another monomer.

Disclosed is also the use of the polymer mixture for preparing a dosage form with a sustained release profile and resistance against the influence of ethanol and the dosage form itself.

Polymer 1

Polymer 1 is a polymer, polymerized from a monomer mixture comprising, essentially comprising to 98% by weight or more, or consisting to 100% out of the monomers

(a1) 70 to 95% by weight of a C₁-C₁₂, C₂-C₁₀ or a C₂-C₈ alkylester of acrylic acid or of methacrylic acid, most preferred 2-Ethylhexyl methacrylate (EHMA) and Ethyl methacrylate, and

(b1) 5 to 30% by weight of a C₂-C₈ alkyl ester of acrylic acid or of methacrylic acid with a quaternary cationic group, preferably a quaternary ammonium group, in the alkyl group, most preferred 2-Trimethylammonium-ethyl-methacrylate-chloride (TMAEMC). The monomers (a1) and (b1) may add up to 98% by weight or more or preferably to 100%.

Most preferred polymer 1 does not contain any further polymerized monomers except for monomers (a1) and (b1).

The terms “monomer (a1)” and “monomer (b1)” shall be understood in the sense of one or more monomers (a1) and one or more monomer (b1) and shall include any kind of mixtures of monomer (a1) and any kind of mixtures of monomer (b1).

Monomer (a1)

The monomer (a1) may be selected from the group of C₁-C₁₂, preferably C₁-C₁₂ or C₂-C₁₀ or most preferred from C₂-C₈ alkyl esters of acrylic acid or of methacrylic acid.

C₁-C₁₂, alkyl esters of acrylic acid or of methacrylic acid are for instance: methyl acrylate, ethyl acrylate, propyl acrylate, iso-propyl acrylate, butyl acrylate, pentyl acrylate, hexyl acrylate, heptyl acrylate, octyl acrylate, nonyl acrylate, decyl acrylate or dodecyl acrylate (lauryl acrylate), methyl methacrylate, ethyl methacrylate, propyl methacrylate, iso-propyl methacrylate, butyl methacrylate, pentyl methacrylate, hexyl methacrylate, heptyl methacrylate, octyl methacrylate, nonyl methacrylate, decyl methacrylate or lauryl methacrylate.

The C₁-C₁₂, C₂-C₁₀ or C₂-C₈ alkyl esters of acrylic acid or of methacrylic acid (a1) are most preferably selected from 2-Ethylhexyl methacrylate (EHMA) and Ethyl methacrylate (EMA), which means 2-Ethylhexyl methacrylate (EHMA) and Ethyl methacrylate (EMA) or both of them.

2-Ethylhexyl methacrylate (EHMA) and Ethyl methacrylate (EMA) may be included as monomers (a1) and (a2). The ratio by weight of 2-Ethylhexyl methacrylate (EHMA): Ethyl methacrylate (EMA) may be preferably in the range from 5:1 to 1:1, from 4:1 to 1:1.

Monomers (b1)

The C₂-C₆ alkyl ester of acrylic acid or of methacrylic acid with a quaternary cationic group, preferably a quaternary ammonium group, in the in the alkyl group (c) may be preferably 2-trimethylammonium-ethyl-methacrylate-chloride (TMAEMC) or 2-trimethylammonium-propyl-methacrylate-chloride (TMAPMC). Most preferred is 2-trimethylammonium-ethyl-methacrylate-chloride (TMAEMC).

Polymer 2

Polymer 2 is a polymer polymerized from a monomer mixture, comprising, essentially comprising to 98% by weight or more, or consisting to 100% out of the monomers

(a2) 70-95% by weight of a C₁-C₁₂, C₂-C₁₀ or a C₂-C₈ alkylester of acrylic acid or of methacrylic acid, and

(c2) 5-30% by weight of a C₂-C₈ or a C₂-C₄ hydroxy-alkylester of acrylic acid or methacrylic acid. The monomers (a2) and (c2) may add up to 98% by weight or more or to 100%.

Most preferred polymer 2 does not contain any further polymerized monomers except for monomers (a2) and (c2).

The terms “monomer (a2)” and “monomer (c2)” shall be understood in the sense of one or more monomers (a2) and one or more monomer (c2) and shall include any mixtures of monomer (a2) and any mixtures of monomer (c2).

Monomer (a2)

The monomer(s) (a2) of polymer 2 may be identical or different to the monomer(s) (a1) of polymer 1.

The monomer (a2) may be selected from the group of C₁-C₁₂, preferably C₁-C₁₂, C₂-C₁₀ or most preferred C₂-C₈ alkyl esters of acrylic acid or of methacrylic acid.

C₁-C₁₂, C₁-C₁₂, C₂-C₁₀ or C₂-C₈ alkyl esters of acrylic acid or of methacrylic acid are for instance methyl acrylate, ethyl acrylate, propyl acrylate, iso-propyl acrylate, butyl acrylate, pentyl acrylate, hexyl acrylate, heptyl acrylate, octyl acrylate, nonyl acrylate, decyl acrylate or dodecyl acrylate (lauryl acrylate), methyl methacrylate, ethyl methacrylate, propyl methacrylate, iso-propyl methacrylate, butyl methacrylate, pentyl methacrylate, hexyl methacrylate, heptyl methacrylate, octyl methacrylate, nonyl methacrylate, decyl methacrylate or lauryl methacrylate.

The C₁-C₁₂, C₂-C₁₀ or C₂-C₆ alkyl esters of acrylic acid or of methacrylic acid (a) are most preferably selected from 2-Ethylhexyl methacrylate (EHMA) and Ethyl methacrylate (EMA), which means 2-ethylhexyl methacrylate (EHMA) and ethyl methacrylate (EMA) or both of them.

Most preferred 2-Ethylhexyl methacrylate (EHMA) and ethyl methacrylate (EMA) are included as monomers (a2). The ratio by weight of 2-ethylhexyl methacrylate (EHMA):ethyl methacrylate (EMA) may be preferably in the range from 5:1 to 1:1, from 4:1 to 1:1.

Monomers (c2)

The monomer (a2) may be selected from the group of C₂-C₆ or C₂-C₄ hydroxy-alkylesters of acrylic acid or methacrylic acid.

C₂-C₆ or C₂-C₄ hydroxy-alkylesters of acrylic acid or methacrylic acid are for instance 2-hydroxyethyl methacrylate (HEMA), 2-hydroxypropyl methacrylate, 3-hydroxypropyl methacrylate, 2,3-dihydroxypropyl methacrylate, 2-hydroxyethyl acrylate, 2-hydroxypropyl acrylate, 3-hydroxypropyl acrylate and 2,3-dihydroxypropyl acrylate or any mixtures thereof.

The C₂-C₆ or C₂-C₄ hydroxy-alkylesters of acrylic acid or methacrylic acid (b) is preferably 2-hydroxyethyl methacrylate (HEMA).

Further Monomers

The polymers 1 and 2, independent from each other, may optionally comprise 2% or less by weight, 0-2% by weight of further vinylically copolymerizable monomers or most preferred not any further vinylically copolymerizable monomers at all.

Minimum Film Forming Temperature (MFFT)

Preferably wherein the minimum film forming temperature of polymer 1 or polymer 2 (MFFT) is 35° C. or lower, 30° C. or lower, 25° C. or lower, 20° C. or lower or 15° C. or lower.

Preferably of polymer 1 or polymer 2 show a minimum film forming temperature (MFFT) of 5 to 35, 8 to 30, 9 to 25 or 10 to 20° C.

As a rule polymers 1 and polymers 2 with identical or with different minimum film forming temperatures (MFFT) may be selected independently from each other and may be combined freely without restriction.

The MFFT may be determined according to the Standard of the International Organisation for Standardization DIN ISO 2115 with the exception of point 6.1 in that the maximum difference of the most distant metering points is set to 50° C.

Midpoint Glass Transition Temperature (T_{mg})

Preferably, the midpoint glass transition temperature (T_{mg}) of polymer 1 is in the range from −10 to 30, preferably from 0 to 20° C.

Preferably, the midpoint glass transition temperature (T_{mg}) of polymer 2 is in the range from 0 to 50, preferably from 20 to 45° C.

As a rule polymers 1 and polymers 2 with identical or with different midpoint glass transition temperatures (T_{mg}) may be selected independently from each other and may be combined freely without restriction.

DSC measurement of the dry polymer substance was conducted according to DIN EN ISO 11357-2 with a heating rate of 20° C./min. The midpoint glass transition temperature T_{mg} was determined by half step height method as described in section 10.1.2 of DIN EN ISO 11357-2.

Molecular weight Mw—Polydispersity Index

Preferably, the weight average molecular weight M_{w} of polymer 1 or polymer 2 is from 50.000 to 200.000, preferably from 60.000 to 120.000 Dalton.

The polydispersity index may be determined by calculation of the M_{w}/Mₙ ratio (weight average molecular weight/number average molecular weight (determined by GPC)). The polydispersity index of the inventive polymer may be in the range from 1.2 to 4.0, 1.3 to 3.0, 1.5 to 2.5 or from 1.6 to 2.3.

Gel permeation chromatography (GPC) is used to determine the number- and weight-average molecular weights (Mₙ, M_{w}) and the polydispersity (D) of the inventive polymers as disclosed according to DIN 55672-1. Equipment consisted of four PSS SDV columns (Mainz, Germany) plus pre-column of the same type, a column oven operating at 35° C., an Agilent (Series 1100, Santa Clara, USA) pump plus RI-detector of the same series. A 0.02 M solution of 2-(diethylamino)ethylamine (DEAEA) in tetrahydrofuran (THF) was used as eluent at a flow rate of 1 ml/min. Samples were dissolved in the eluent at concentrations of 2 mg/ml. For each measurement 100 μL polymer solution was injected. The values for Mn and Mw were calculated based on calibration curves generated by Poly(methyl methacrylate) standards.

EUDRAGIT® reference samples were measured using the eluent N,N-dimethylacetamide (DMAc). A method for EUDRAGIT® RL/RS is described in more detail by Adler M. et al. (e-Polymers, ISSN (Online) 1618-7229, ISSN (Print) 2197-4586, DOI: https://doi.org/10.1515/epoly.2005.5.1.602). A method for EUDRAGIT® NM is described in more detail by Adler M. et al. (e-Polymers, ISSN (Online) 1618-7229, ISSN (Print) 2197-4586, DOI: https://doi.org/10.1515/epoly.2004.4.1.608).

### PREFERRED EMBODIMENT

In a preferred embodiment the polymer mixture may be a mixture from a

polymer 1, polymerized from a monomer mixture consisting of the monomers

(a1) 70 to 95, preferably 85 to 95% by weight of 2-ethylhexyl methacrylate and 2-ethyl methacrylate at a ratio of from 3:1 to 1:1.

(b1) 5 to 30, preferably 5-15% by weight of 2-trimethylammonium-ethyl-methacrylate-chloride and a polymer 2, polymerized from a monomer mixture consisting of the monomers

(a2) 70 to 95, preferably 85 to 95% by weight of 2-ethylhexyl methacrylate and 2-ethyl methacrylate at a ratio from 3:1 to 1:1.

(c2) 5-30, preferably 5-15% by weight of 2-hydroxyethyl methacrylate, wherein the polymer mixture is comprising polymer 1 to polymer 2 at a ratio by weight of from 2:1 to 1:2, and wherein polymer 1 to polymer 2 add up to 100%.

Preferably, the polymers 1 and/or 2 of the preferred embodiment may have one, two or three of the following properties in any possible combination:
The midpoint glass transition temperature (T_{mg}) of polymer 1 is in the range from −10 to 30, preferably from 0 to 20° C.
The midpoint glass transition temperature (T_{mg}) of polymer 2 is in the range from 0 to 50, preferably from 20 to 45° C.
The weight average molecular weight Mw of polymer 1 or polymer 2 is from 50.000 to 200.000, preferably from 60.000 to 120.000 Dalton.
The minimum film forming temperature of polymer 1 or polymer 2 (MFFT) may be 5 to 35° C.

Process for Preparing the Polymer

A process for preparing a polymer mixture may be characterized in that the polymer 1 and polymer 2 are polymerized independently from each other from their monomers in the presence of a polymerization-initiator and optionally a Chain-transfer agent by bulk polymerization, suspension polymerization or emulsion polymerization and are mixed subsequently.

The polymer 1 and the polymer 2 may be prepared by radical polymerisation of the corresponding monomers in the presence of polymerisation initiators such as ammonium-peroxodisulfate.

A Chain transfer agent may be added to improve the process stability and reproducibility of the molecular weight (Mw). However the Chain-transfer agent may be omitted in many cases, without affecting the properties according to the invention.

Preparation methods for the polymer are known to the expert in the field. Typically emulsion polymerization, solution polymerization or bulk polymerization will be applied; the preferred preparation of the polymer is by emulsion polymerization.

If emulsion polymerization is used, the operation may advantageously be carried out by the monomer emulsion feed process or the monomer feed process, respectively. For this, water is heated to the reaction temperature in the polymerization reactor. Surfactants/emulsifiers and/or initiators may be added at this stage. Then, depending on the mode of operation, a monomer, a monomer mixture or an emulsion of either are fed to the reactor. This dosed liquid may contain initiators and/or surfactants or the initiator and/or the surfactant may be dosed in parallel.

Alternatively, all monomers can be charged into the reactor, before adding the initiator. This method is often referred to as batch process.

It is also possible to do a combination of both processes, by polymerizing a part of the monomers in the manner of a batch process, and feeding the other part afterwards.

As known to the expert in the field, the type of process and mode of operation may be chosen, to achieve the desired particle size, sufficient dispersion stability, a stable production process and so on.

Emulsifiers

Emulsifiers which may be used are especially anionic and non-ionic surfactants. The amount of emulsifier used is generally not more than 5% by weight, preferably in the range of 0.1 to 4% by weight, based on weight of the monomer mixture.

Typical emulsifiers are for example alkyl sulfates (e.g. sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and others.

Polymerization Initiators Beside those initiators conventionally used in emulsion polymerization (e.g. per-compounds, such as ammonium peroxodisulfate (APS)) redox systems, such as sodium disulphite-APS-iron can be applied. Also water soluble azo-initiators may be applied and/or a mixture of initiators can be used. The amount of polymerization initiator, most preferred ammonium peroxodisulfate (APS), for the polymerisation of polymer 1 or polymer 2 may be around 0.005 to 0.5, 0.05 to 0.2, 0.01 to 0.1% by weight, based on total weight of the corresponding monomers.

Chain-Transfer Agents

Chain-transfer agents are well known to the skilled person and used for controlling the molecular weight and weight distribution in a polymerization process.

A Chain-transfer agent may be added to the monomer mixture of polymer 1 or polymer 2 before or during the polymerization. Up to 5, up to 4, up to 3, up to 2, up to 1% by weight or 0.05 to 5, 0.1 to 4, 0.2 to 3, 0.25 to 2, 0.1 to 1, 0.05 to 0.5, 0.1 to 0.4% by weight of a Chain transfer agent, calculated on the total weight (100%) of the monomers, may be added to the monomer mixture. It is also possible to add not any Chain-transfer agent at all (0%).

A suitable chain-transfer agent may be 2-ethylhexylthioglycolat (TGEH) or n-butylmercaptan, n-dodecylmercaptan or 2-mercaptoethanol or any mixtures thereof. Most preferred is 2-ethylhexylthioglycolat (TGEH).

Polymerization Temperature

A suitable polymerization temperature may be in the range of 25 to 120, 30 to 100, 50 to 95° C.

The polymerization temperature may depend on the initiators within certain limits. For example, if APS is used it is advantageous to operate in the range from 60 to 90° C.; if redox systems are used it is also possible to polymerize at lower temperatures, for example in the range of 25 to 45° C., for instance at 30° C.

The average particle size of the polymer particles produced in the emulsion polymerization may range from 10 to 1000, 20 to 500 or 50 to 250 nm. The average particle size of the polymer particles may be determined by methods well known to a skilled person for instance by the method of laser diffraction. The particle size may be determined by laser diffraction, using a Mastersizer 2000 (Malvern). The values can be indicated as particle radius rMS [nm], which is half of the median of the volume based particle size distribution d(v,50).

The obtained dispersion can directly be used to prepare the coating suspension, or—in rare cases—be used as coating suspension without even adding further ingredients.

The dispersion can also be dried, preferably by spray drying, freeze drying or coagulation. Thus a solid can be obtained, which offers certain advantages with regard to handling and logistics.

The dried polymer may then be transferred into a coating suspension by redispersing the solid in water, e.g. (where required) by the use of a high shear mixer.

The dried polymer may also be dissolved in a solvent, e.g. an organic solvent, to prepare a coating solution.

If coating with coating solutions is preferred, the preparation of the polymer by solution polymerization or bulk polymerization may be a good option, too.

Sustained or Extended Release Pharmaceutical or Nutraceutical Composition

The composition as disclosed herein is preferably a pharmaceutical or nutraceutical dosage form, preferably a sustained release or extended release pharmaceutical or nutraceutical dosage form.

The sustained or extended release of the pharmaceutical or nutraceutical active ingredient may be defined in that the active ingredient release under in-vitro conditions after 2 hours at pH 1.2 in simulated gastric fluid according to USP (for instance USP 32) and subsequent change of the medium to buffered medium of pH 6.8 according to USP may be for instance in the range of 20 to 98, 30 to 90, 40 to 80% in a total time of 4 to 12 or 4 to 8 or 6 to 10 hours, including the 2 hours of the pH 1.2 phase.

Ethanol Resistant Composition

The composition as disclosed herein is an ethanol (EtOH) resistant composition, preferably an ethanol (EtOH) resistant pharmaceutical or nutraceutical composition.

Ethanol resistant shall mean that the release of a biologically active ingredient, preferably a pharmaceutical or nutraceutical active ingredient, under in-vitro conditions at pH 1.2 for 2 hours in simulated gastric fluid according to USP and subsequent buffer pH 6.8 without the addition of ethanol does not differ by more than plus/minus 20, preferably plus/minus 10% (absolute percentage) in the same media but with the addition of 5, 10, 20 or 40% (w/w) ethanol in the pH 1.2 medium only.

To give an example if the release rate of the pharmaceutical or nutraceutical active ingredient is in the medium without ethanol for instance 60% then the active ingredient release in the same medium with ethanol shall be in the range from 40 to 80% (+/−20% deviation).

Ethanol resistant dosage forms as defined herein are formulations with release kinetics in pH 1.2 medium and subsequent pH 6.8 medium not significantly affected by the presence of ethanol in a pH 1.2 medium. Ethanol resistance may be an important registration requirement in the near future. Conventional pharmaceutical compositions if coated or uncoated are usually not resistant to alcohol at all. An ethanol resistant formulation is sometimes also called a rugged formulation.

Resistance against the influence of ethanol (ethanol resistant dosage form) may be defined in that the release profile determined under in-vitro conditions at pH 1.2 and/or at pH 6.8 in a buffered medium according to USP with the addition of 40% (w/w) ethanol is not accelerated by more than 20%, preferably by not more than 10%, and not delayed by more than 20%, preferably by not more than 10%, under the influence of the 40% ethanol containing medium in comparison to a release profile determined in the same medium without ethanol. Generally an acceleration of a release profile is more critical than a delay. Therefore, the upper limit for an acceleration of the release profile is preferably not more than 10%, more preferably not more than 5%, even more preferably there is no acceleration of the release profile at all.

Depending on the certain dosage form the applicable conditions of the USP test may vary for instance if the paddle or basket method has to be used or the stirring has to be 50, 100 or 150 rpm. For the determination of the ethanol resistance it does not matter which USP test is applied for the certain pharmaceutical composition as long as it is the relevant test for the certain pharmaceutical (or nutraceutical) composition and the test conditions with and without ethanol are the same.

Resistance against the influence of ethanol in the sense of the present invention shall be tested in a relevant period of the release of the active ingredient, where meaningful results can be expected. The period which is meaningfully chosen is from or between 10 to 80% of the total dosage release in the medium without ethanol. In this period the resistance against the influence of ethanol shall be determined at a number n of at least n=3, but preferably more than 3, for instance n=4, 5, 6, 7, 8, 9, 10, 11 or 12 uniformly distributed test points. The number of meaningfully chosen test points depends on the total time period of the release profile from or between 10 to 80% of the total dosage release. The longer the time period the more uniformly distributed test points can be chosen meaningful. The first test point should be the first full hour or half hour time point at or after the 10% release point. The last test point should be at the last full hour or half hour time point at or before the 80% release point. The other test point or test points should be in the middle (n=3) or uniformly distributed (n>3) at full hour or half hour time points at or in between the 10 and 80% release phase. The percentage of acceleration or delay is calculated by the arithmetic mean (arithmetic average) of the n values to give the arithmetic mean release.

The term “and/or” in “under in-vitro conditions at pH 1.2 and/or at pH 6.8” means that there may be different meaningful conditions for different pharmaceutical (or nutraceutical) compositions. Resistance against the influence of ethanol shall be determined only in a relevant period of the release of the active ingredient.

Sustained release pharmaceutical compositions have periods of the release of the active ingredient for instance from 6 to 12 or even more hours, with usually more than 10% release within the first two hours at pH 1.2. In this case it is meaningful to test under in-vitro conditions at pH 1.2 and at pH 6.8.

The percentages of acceleration or delay under the influence of the 5, 10, 20 or 40% ethanol containing pH 1.2 medium are calculated by subtraction of corresponding single release values and the calculation of the arithmetic average thereof. The n release values taken from the media (pH 1.2 and subsequent pH 6.8) with ethanol in the pH 1.2 medium are subtracted by the corresponding n release values from the media without ethanol in the pH 1.2 medium and the arithmetic average of the differences is calculated. A positive result stands for an acceleration of the release; a negative result stands for a delayed release.

A dosage form which fulfils these conditions can be considered to be resistant against critically accelerated release or delay of the active compound by thoughtlessness or by addictive behaviour of the patients with respect to the use of ethanol or ethanol-containing drinks. This situation relates essentially to the simultaneous or subsequent consumption of an alcoholic drink together with the taking of the controlled release pharmaceutical form, such that the pharmaceutical form is exposed to a strong ethanol-containing medium in the stomach or intestine.

However, the purpose of the present invention is expressively not to stimulate, to promote or to make possible the consumption of ethanol-containing drinks together with delayed-release pharmaceutical forms, but to alleviate or to avoid the possibly fatal consequences of intentional or inadvertent misuse or abuse.

### Calculation Example 1

If the arithmetic average calculated from the active ingredient release in the medium with ethanol and without ethanol is 8% (=plus 8%), then there is an acceleration caused by the influence of ethanol of 8%. In this case the controlled release pharmaceutical composition is regarded to be resistant against the influence of ethanol because it is within the limit of not more than 20% acceleration.

### Calculation Example 2

If the arithmetic average calculated from the active ingredient release in the medium with ethanol and without ethanol is minus 23% (−23%), then there is a delay caused by the influence of ethanol of 23%. In this case the controlled release pharmaceutical composition is not regarded to be resistant against the influence of ethanol because it is out of the limit of not more than 20% delay.

Biologically Active Ingredients

The biologically active ingredient may be preferably a pharmaceutical active ingredient and/or a nutraceutical active ingredient.

Pharmaceutical Active Ingredients

The invention is preferably useful for pharmaceutical dosage forms, preferred for sustained release formulated pharmaceutical dosage forms.

Therapeutical and chemical classes of active ingredients used in sustained release formulated coated pharmaceutical dosage forms are for instance analgetics, antibiotics or anti-infectives, antibodies, antiepileptics, antigens from plants, antirheumatics, betablocker, benzimidazole derivatives, beta-blocker, cardiovascular drugs, chemotherapeutics, CNS drugs, digitalis glycosides, gastrointestinal drugs, e.g. proton pump inhibitors, enzymes, hormons, liquid or solid natural extracts, oligonucleotides, peptidhormon proteins, therapeutical bacteria, peptides, proteins (metal)salt f.e. aspartates, chlorides, orthates, urology drugs, vaccines

Further examples of drugs for sustained controlled release may be: acamprosat, aescin, amylase, acetylsalicylic acid, adrenalin, 5-amino salicylic acid, aureomycin, bacitracin, balsalazine, beta carotene, bicalutamid bisacodyl, bromelain, bromelain, budesonide, calcitonin, carbamacipine, carboplatin, cephalosporins, cetrorelix, clarithromycin,chloromycetin, cimetidine, cisapride, cladribine, clorazepate, cromalyn, 1-deaminocysteine-8-D-arginine-vasopressin, deramciclane, detirelix, dexlansoprazole, diclofenac, didanosine, digitoxin and other digitalis glycosides, dihydrostreptomycin, dimethicone, divalproex, drospirenone,duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormon (hGH), ibuprofen, ilaprazole, insulin, Interferon, Interleukin, Intron A, ketoprofen, lansoprazole, leuprolidacetat lipase, lipoic acid, lithium, kinin, memantine, mesalazine, methenamine, milameline, minerals, minoprazole, naproxen, natamycin, nitrofurantion, novobiocin, olsalazine, omeprazole, orothates, pancreatin, pantoprazole, parathyroidhormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, thrypsine, valproic acid, vasopressin, vitamins, zinc, including their salts, derivatives, polymorphs, isomorphs, or any kinds of mixtures or combinations thereof.

Further examples for pharmaceutical active ingredients may be caffeine citrate, metoprolol succinate and theophylline.

Nutraceutical Active Ingredients

The invention is preferably useful for nutraceutical dosage forms, preferred for sustained release formulated nutraceutical I dosage forms.

Nutraceuticals are well known to the skilled person. Nutraceuticals are often defined as extracts of foods claimed to have medical effects on human health. Thus nutraceutical active ingredients may display pharmaceutical activities as well: Examples for nutraceutical active ingredients may be resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Thus it is clear that many substances listed as nutraceuticals may also be used as pharmaceutical active ingredients.

Depending on the territory, the specific application, the local authority legislation and classification, the same substance may be listed as a pharmaceutical or as a nutraceutical active ingredient respectively as a pharmaceutical or a nutraceutical composition or even both. Thus it is evident to a skilled person that there is a broad overlap between the terms pharmaceutical or a nutraceutical active ingredient respectively a pharmaceutical or a nutraceutical composition.

The invention is preferably useful for nutraceutical dosage forms.

Nutraceuticals or nutraceutical active ingredients are sometimes defined as extracts of foods claimed to have medical effects on human health.

Nutraceuticals or nutraceutical active ingredients may also include probiotics and prebiotics. Probiotics are living microorganisms believed to support human or animal health when consumed, for example certain strains of the genera Lactobacillus or Bifidobacterium. Prebiotics are nutraceuticals or nutraceutical active ingredients that induce or promote the growth or activity of beneficial microorganisms in the human or animal intestine.

The nutraceutical active ingredient may be usually contained in a medical format such as capsule, tablet or powder in a prescribed dose. Examples for nutraceuticals are resveratrol from grape products or pro-anthocyanines from blueberries as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, antioxidants, alpha-linoleic acid from flax seed, beta-carotene from marigold petals or antocyanins from berries. Sometimes the expression neutraceuticals or nutriceuticals are used as synonyms for nutraceuticals.

Dosage Form

Disclosed is a dosage form, comprising a core, comprising a biologically active ingredient, and a coating layer onto the core, wherein the coating layer is comprising the polymer mixture as disclosed herein and optionally pharmaceutical or nutraceutical excipients.

The coating layer of the dosage form as disclosed may comprise 25-100, preferably 30 to 80% by weight of the polymer mixture and 0 to 75, preferably 20 to 70% by weight of pharmaceutical or nutraceutical excipients. The dosage form may also comprise only the polymer mixture and any pharmaceutical or nutraceutical excipients.

The pharmaceutical or nutraceutical acceptable excipients may be selected from the groups of antioxidants, brighteners, binding agents, flavouring agents, flow aids, fragrances, glidants, penetration-promoting agents, pigments, plasticizers, cellulosic polymers, pore-forming agents or stabilizers or any combinations thereof.

The dosage form comprises a core, which comprises a biologically active ingredient. Depending on the intended use in the pharmaceutical and/or in the nutraceutical field, the biologically active ingredient may be a pharmaceutical active ingredient and/or a nutraceutical active ingredient. A pharmaceutical active ingredient or a nutraceutical active ingredient is a biologically active ingredient comprised in an oral dosage form with an intended application or use in the field of pharmaceuticals and/or nutraceuticals.

The intention of the application of the dosage form in the pharmaceutical field is usually the therapy of diseases of man or animals. The intention of the application of the dosage form in the nutraceutical field is usually the prevention of diseases and general support of vitality and health of man or animals.

The dosage form is preferably an oral dosage form and depending on the intended use a pharmaceutical or nutraceutical dosage form. The dosage form comprises, comprises essentially or is consisting of a core and a coating layer. The core comprises a biologically active ingredient. The coating is located directly or indirectly onto the core and comprises the polymer as disclosed herein and optionally excipients, such as pharmaceutical or nutraceutical acceptable excipients. The dosage form, respectively the pharmaceutical or nutraceutical dosage form is intended to be used as an oral dosage form in the field of pharmaceuticals and/or nutraceuticals.

The dosage form may be a tablet, a minitablet, a pellet, a granule, a sachet, a capsule, filled with coated pellets or with powder or with granules, or a coated capsule.

Pellets or granules may be used as cores or in compressed tablets. As a rough estimation, pellets may have a size in the range of 50 to 1500, 250 to 1250 μm (average diameter), while coated tablets may have a size in the range of above 1000 μm up to 25 mm (diameter or length). As a rule one can say the smaller the size of the pellet cores are, the higher the pellet coating weight gain needed. This is due to the comparably higher surface area of pellets compared to tablets.

The term pellet-containing tablet or compressed tablet is well known to a skilled person. Such a tablet may have a size of around 5 to 25 mm for instance. Usually, defined pluralities of small active ingredient containing pellets are compressed therein together with binding excipients to give the well-known tablet form. After oral ingestion and contact with the body fluid the tablet form is disrupted and the pellets are set free. The compressed tablet combines the advantage of the single dose form for ingestion with the advantages of a multiple form, for instance the dosage accuracy. In tablets containing comparably low amounts of excipients, preferably talcum but also other excipients, may be used in contrast to pellets.

The term minitablet is well known to the skilled person. A minitablet is smaller than the traditional tablet and may have a size of around 1 to 4 mm. The minitablet is, like a pellet, a single dosage form to be used in multiple dosages. In comparison to pellets, which may be in the same size, minitablets usually have the advantage of having more regular surfaces which can be coated more accurately and more uniformly. Minitablets may be provided enclosed in capsules, such as gelatine capsules. Such capsules disrupt after oral ingestion and contact with the gastric or intestinal fluids and the minitablets are set free. Another application of minitablets is the individual fine adjustment of the active ingredient dosage. In this case the patient may ingest a defined number of minitablets directly which matches to the severeness of the disease to cure but also to his individual body weight. A minitablet is different from pellet-containing compressed tablet as discussed above.

The term sachet is well known to the skilled person. It refers to small sealed package which contains the active ingredient often in pellet containing liquid form or also in dry pellet or powder form. The sachet itself is only the package form and is not intended to be ingested. The content of the sachet may be dissolved in water or as an advantageous feature may be soaked or ingested directly without further liquid. The latter is an advantageous feature for the patient when the dosage form shall be ingested in a situation where no water is available. The sachet is an alternative dosage form to tablets, minitablets or capsules.

The term capsule is well known to the skilled person. A capsule is like the sachet a container for pellets containing liquids or also dry pellets or powders. However in contrast to the sachet the capsule consists of pharmaceutically acceptable excipients such as gelatine or hydroxypropylmethylcellulose (HPMC) and is intended to be ingested like a tablet. The capsules disrupts after oral ingestion and contact with the gastric or intestinal fluids and the contained multiple units are set free. Capsules for pharmaceutical purposes are commercially available in different standardized sizes.

Core

The core may be a pellet, a granule, a tablet or a capsule. The core is coated with a coating layer comprising the polymer as disclosed and optionally excipients, preferably pharmaceutical or neutraceutical acceptable excipients. The core may be an active ingredient containing tablet, a pellet containing compressed tablet, a mini tablet or a capsule, which may be filled with pellets.

The core may comprise an uncoated pellet, a neutral carrier pellet, for instance a sugar sphere or non-pareilles, on top of which the biologically active ingredient is bound in a binder, such as lactose or polyvinylpyrrolidone. The layer with the biologically active ingredient is considered herein as part of the core. The core may as well comprise an uncoated pellet consisting of a crystallized biologically active ingredient.

The core may comprise 1 to 100, 2 to 90, 5 to 85, 10 to 70, 15 to 50% by weight of the biologically active ingredient. The core may comprise 0 to 99, 10 to 98, 15 to 95, 30 to 90 or 50 to 85% by weight of excipients, preferably pharmaceutical or nutraceutical acceptable excipients. The biologically active ingredient and the excipients may add up to 100%.

In the case of a core which is an uncoated pellet, the coating with the ethanol resistance conferring coating layer has the functions of providing at first the desired release properties function to the pharmaceutical composition and secondly to provide resistance against the influence of ethanol.

Coating Layer

The core of the dosage form is coated with a coating layer comprising the polymer as disclosed and optionally excipients, preferably pharmaceutical or neutraceutical acceptable excipients.

The coating layer may comprise at least 2, at least 5, at least 10, at least 20, at least 30, at least 40, at least 50, at least 60, at least 70, at least 80, at least 90 or 100% by weight of the polymer as disclosed and claimed herein. The coating layer may comprise 2 to 100, 5 to 98, 10 to 90, 12 to 80, 15 to 70, 18 to 60 or 20 to 50% by weight of the polymer mixture as disclosed herein.

The coating layer may comprise up to 10, up to 20, up to 30, up to 40, up to 50, up to 60, up to 70, up to 80, up to 90, up to 95, up to 98% by weight of exipients, preferably pharmaceutical or nutraceutical excipients (pharmaceutical or neutraceutical acceptable excipients). The coating layer may comprise to 98, 2 to 95, 10 to 90, 20 to 88, 30 to 85, 40 to 82 or 50 to 80% by weight of excipients. The polymer and the optionally comprised pharmaceutical or neutraceutical excipients may add up to 100%.

The dosage form or the coating layer of the dosage form may optionally be further defined in that a polymer, polymerized from a monomer mixture comprising

(a) 70 to 95% by weight of a C₁-C₁₂ alkylester of acrylic acid or of methacrylic acid,
(b) 2.5 to 15% by weight of a C₂-C₆ hydroxy-alkylester of acrylic acid or methacrylic acid,
(c) 2.5 to 15% by weight of a C₂-C₈ alkyl ester of acrylic acid or of methacrylic acid with a quaternary cationic group in the alkyl group is not contained.

Excipients

Excipients are well known to a skilled person and formulated along with the biologically active ingredient and/or with the polymer mixture as disclosed herein. All excipients used must be toxicologically safe and be used in pharmaceuticals or nutraceuticals without risk for patients or consumers. Excipients may be added for practical reasons, for instance as processing aids, to avoid stickiness or to add color. The addition of excipients shall not negatively affect or alter the ethanol resistance properties as disclosed.

The dosage form may comprise excipients, preferably pharmaceutical or nutraceutical acceptable excipients, that may be selected from the group of antioxidants, brighteners, binding agents, flavouring agents, flow aids, fragrances, glidants, penetration-promoting agents, pigments, plasticizers, polymers different from polymer 1 and polymer 2, for instance cellulosic polymers or other neutral polymers or copolymers, pore-forming agents or stabilizers or combinations thereof. The pharmaceutical or nutraceutical acceptable excipients may be comprised in the core and/or in the coating layer comprising the polymer mixture as disclosed. A pharmaceutical or nutraceutical acceptable excipient is an excipient, which is allowed to be used for the application in the pharmaceutical or nutraceutical field.

The coating layer may comprise up to 98, up to 95, up to 90, up to 80, up to 70, up to 50, up to 60, up to 50, up to 40, up to 30, up to 20, up to 10% by weight or not any (0%) excipients at all, respectively pharmaceutical or nutraceutical acceptable excipients. Preferably, except for the inventive polymer mixture, no further polymers are present in the coating layer.

Plasticizers

Plasticizers achieve through physical interaction with a polymer a reduction in the glass transition temperature and promote film formation, depending on the added amount. Suitable substances usually have a molecular weight of between 100 and 20 000 and comprise one or more hydrophilic groups in the molecule, e.g. hydroxyl, ester or amino groups.

Examples of suitable plasticizers are alkyl citrates, glycerol esters, alkyl phthalates, alkyl sebacates, sucrose esters, sorbitan esters, diethyl sebacate, dibutyl sebacate, propylenglycol and polyethylene glycols 200 to 12 000. Preferred plasticizers are triethyl citrate (TEC), acetyl triethyl citrate (ATEC), diethyl sebacate and dibutyl sebacate (DBS). Mention should additionally be made of esters, which are usually liquid at room temperature, such as citrates, phthalates, sebacates or castor oil. Esters of citric acid and sebacinic acid are preferably used.

Addition of the plasticizers to the formulation can be carried out in a known manner, directly, in aqueous solution or after thermal pre-treatment of the mixture. It is also possible to employ mixtures of plasticizers. However, since the polymer as disclosed herein shows a minimum film forming temperature (MFFT) of 35° C. or lower, it is possible to apply the polymer coating, for instance from an aqueous polymer dispersion, without the addition of a plasticizer. Thus, the coating layer may comprise up to 25, up to 20, up to 15, up to 10, up to 5, but preferably less than 5% by weight calculated on the polymer of a plasticizer or any (0%) plasticizer at all.

Fillers

Standard fillers are usually added to the inventive formulation during processing to coating and binding agents. The quantities introduced and the use of standard fillers in pharmaceutical coatings or overlayers is familiar to those skilled in the art. Examples of standard fillers are release agents, pigments, stabilizers, antioxidants, pore-forming agents, penetration-promoting agents, brighteners, fragrances or flavouring agents. They are used as processing adjuvants and are intended to ensure a reliable and reproducible preparation process as well as good long-term storage stability, or they achieve additional advantageous properties in the pharmaceutical form. They are added to the polymer formulations before processing and can influence the permeability of the coatings. This property can be used if necessary as an additional control parameter.

Glidants (Release Agents):

Glidants or release agents usually have lipophilic properties and are usually added to spray suspensions. They prevent agglomeration of cores during film formation. Suitable glidants are talc, Mg- or Ca-stearate, ground silica, kaolin or nonionic emulsifiers with an HLB value of between 2 and 8. Standard proportions for use of release agents in the inventive coating and binding agents range between 0.5 and 100% by weight relative to polymer.

In a particularly advantageous embodiment, the glidant or release agent is added in concentrated form as the outer layer. Application takes place in the form of powder or by spraying from aqueous suspension with 5 to 30% (weight/weight (w/w)) solid content. The necessary concentration is lower than for incorporation into the polymer layer and amounts to 0.1 to 2% by weight relative to the weight of the dosage form.

The coating layer of the dosage form may for instance comprise 20-80, preferably 30-70% by weight of the polymer mixture as disclosed and 20-80, 30-70% by weight of talc. The polymer mixture and talc may add up to 100% by weight.

Pigments:

As a rule pigments, for instance aluminum oxide or iron oxide pigments, are used in dispersed form, rarely in solute form. Titanium dioxide may be used as a whitening pigment. Standard proportions for use of pigments range are around 10 to 200, 20 to 200% by weight relative to the polymer mixture. Because of the high pigment-binding capacity of the polymer mixture, proportions up to 200% by weight calculated on the polymer mixture can be easily processed.

In a particularly advantageous embodiment, the pigment may be used directly in concentrated form as an outer top coat layer. Application takes place in the form of powder or by spraying from aqueous suspension with 5 to 35% (w/w) solid content. The necessary concentration is lower than for incorporation into the polymer layer and amounts to about 0.1 to 2% by weight relative to the weight of the dosage form.

Process for Preparing the Dosage Form

A suitable process for preparing the dosage form as disclosed herein may be by forming a core comprising the active ingredient by direct compression, compression of dry, wet or sintered granules, by extrusion and subsequent rounding off, by wet or dry granulation, by direct pelleting or by binding powders onto active ingredient-free beads or neutral cores or active ingredient-containing particles or pellets and by applying the coating layer in the form of aqueous dispersions or organic solutions in spray processes or by fluidized bed spray granulation. The water content of aqueous dispersions comprising the polymer mixture and optionally excipients may be in the range of 50 to 95, 60 to 85 or 65 to 80% by weight. The polymer content of an aqueous dispersion may be in the range of 5 to 50, 15 to 40 or 20 to 35% by weight.

Use

Disclosed is also the use, respectively a method of use, of the polymer mixture as disclosed for preparing a dosage form with a sustained release profile and resistance against the influence of ethanol.

Top Coats and Sub Coats

The dosage form according to the invention may be further coated with a sub coat or a top coat or both.

A sub coat may be located between the core and the coating layer, comprising the polymer as disclosed. A sub coat may have the function to separate substances of the core from substances of the controlling layer, which may be incompatible with each other. The sub coat has essentially no influence on the active ingredient release characteristics. A subcoat is preferably essentially water-soluble, for instance it may consist of substances like hydroxypropyl-methyl-cellulose (HPMC) as a film former. The average thickness of the sub coat layer is very thin, for example not more than 15 μm, preferably not more than 10 μm.

A top coat is also preferably water-soluble or essentially water-soluble. A top coat may have the function of colouring the pharmaceutical or nutraceutical form or protecting from environmental influences for instance from moisture during storage. The top coat may consist out of a binder, for instance a water-soluble polymer like a polysaccharide or HPMC, or a sugar compound like saccharose. The top coat may further contain pharmaceutically or nutraceutically acceptable excipients like pigments or glidants in high amounts. The topcoat has essentially no influence on the release characteristics.

Items

The invention is concerned with the following items:
1. Polymer mixture, comprising 10 to 90% by weight of a polymer 1 and 10 to 90% by weight a polymer 2, wherein the weight percentages of polymer 1 and polymer 2 add up to 100%, and wherein the polymer 1 is a
   polymer, polymerized from a monomer mixture comprising the monomers
   (a1) 70-95% by weight of a C₁-C₁₂ or preferred a C₂-C₈ alkylester of acrylic acid or of methacrylic acid, and
   (b1) 5-30% by weight of a C₂-C₈ alkyl ester of acrylic acid or of methacrylic acid with a quaternary cationic group in the alkyl group, and wherein the polymer 2 is a polymer polymerized from a monomer mixture comprising the monomers
   (a2) 70-95% by weight of a C₁-C₁₂ or preferred a C₂-C₈ alkylester of acrylic acid or of methacrylic acid, and
   (c2) 5-30% by weight of a C₂-C₆ hydroxy-alkylester of acrylic acid or methacrylic acid.
2. Polymer mixture according to item 1 wherein the C₁-C₁₂ or C₂-C₈ alkyl ester of acrylic acid or of methacrylic acid (a1) or (a2) is 2-Ethylhexyl methacrylate (EHMA) or Ethyl methacrylate (EMA) or a mixture thereof.
3. Polymer mixture according to item 1 or 2, wherein 2-Ethylhexyl methacrylate (EHMA) and Ethyl methacrylate (EMA) are comprised in (a1) or in (a2) or in both are comprised at a ratio by weight from 5:1 to 1:1, preferably from 4:1 to 1:1.
4. Polymer mixture according to one or more items 1 to 3, wherein the C₂-C₆ alkyl ester of acrylic acid or of methacrylic acid with a quaternary cationic group in the alkyl group (b1) is 2-Trimethylammonium-ethyl-methacrylate-Chloride (TMAEMC) or 2-Trimethylammonium-propyl-methacrylate-Chloride (TMAPMC).
5. Polymer according to one or more items 1 to 4, wherein the C₂-C₆ hydroxy-alkylester of acrylic acid or of methacrylic acid (c2) is selected from 2-Hydroxyethyl methacrylate (HEMA), 2-Hydroxypropyl methacrylate, 3-Hydroxypropyl methacrylate, 2,3-Dihydroxypropyl methacrylate, 2-Hydroxyethyl acrylate, 2-Hydroxypropyl acrylate, 3-Hydroxypropyl acrylate and 2,3-Dihydroxypropyl acrylate or any mixtures thereof.
6. Polymer mixture according to one or more items 1 to 5, wherein in polymer 1 the monomers (a1) and (b1) add up to 97.5% by weight or more, preferably to 98 to 100% by weight, and most preferred to 100%, and wherein in polymer 2, independent from polymer 1, the monomers (a2) and (c2) add up to more than 97.5% by weight, preferably to 98 to 100% by weight, most preferred to 100%.
7. Polymer mixture according to one or more items 1 to 6 and wherein the polymers 1 and 2, independent from each other, optionally comprise 0-2% by weight of further vinylically copolymerizable monomers or most preferred not any further vinylically copolymerizable monomers at all.
8. Polymer mixture according to one or more items 1 to 7, wherein the minimum film forming temperature of polymer 1 or polymer 2 (MFFT) or both is 35° C. or lower, 5 to 35, 8 to 30, 9 to 25 or 10 to 20° C.
9. Polymer mixture according to one or more items 1 to 8, wherein the midpoint glass transition temperature (T_{mg}) of polymer 1 is in the range from −10 to 30, preferably from 0 to 20° C.
10. Polymer mixture according to one or more items 1 to 9, wherein the midpoint glass transition temperature (T_{mg}) of polymer 2 is in the range from 0 to 50, preferably from 20 to 45° C.
11. Polymer mixture according to one or more items 1 to 10, comprising a ratio by weight of polymer 1 to polymer 2 of from 5:1 to 1:5, preferably from 2:1 to 1:2.
12. Polymer mixture according to one or more items 1 to 11, wherein the weight average molecular weight Mw of polymer 1 or polymer 2 is from 50.000 to 200.000, preferably from 60.000 to 120.000 Dalton.
13. Dosage form, comprising a core, comprising a biologically active ingredient, and a coating layer onto the core, wherein the coating layer is comprising a polymer mixture according to any of items 1 to 12 and optionally pharmaceutical or nutraceutical excipients.
14. Dosage form according to item 13, wherein the coating layer comprises 25 to 100, preferably 30 to 80% by weight of the polymer mixture and 0 to 75, preferably 20 to 70% by weight of pharmaceutical or nutraceutical excipients or not any pharmaceutical or nutraceutical excipients at all.
15. Dosage form according to item 13 or 14, wherein the pharmaceutical or nutraceutical acceptable excipients are selected from the group of antioxidants, brighteners, binding agents, flavouring agents, flow aids, fragrances, glidants, penetration-promoting agents, pigments, plasticizers, polymers different from polymer 1 and polymer 2, for instance cellulosic polymers or neutral (meth)acrylate copolymers, pore-forming agents or stabilizers or any combinations thereof.
16. Dosage form according to one or more items 13 to 15, which does not contain a polymer, polymerized from a monomer mixture comprising
   (a) 70 to 95% by weight of a C₁-C₁₂ alkylester of acrylic acid or of methacrylic acid,
   (b) 2.5 to 15% by weight of a C₂-C₆ hydroxy-alkylester of acrylic acid or methacrylic acid, and
   (c) 2.5 to 15% by weight of a C₂-C₈ alkyl ester of acrylic acid or of methacrylic acid with a quaternary cationic group in the alkyl group.
17. Process for preparing a polymer mixture according to one or more of items 1-12, wherein the polymer 1 and the polymer 2 are polymerized independently from each other from their monomers in the presence of a polymerization-initiator and optionally a Chain-transfer agent by bulk polymerization, suspension polymerization or emulsion polymerization and are mixed subsequently to give the polymer mixture.
18. Process according to item 17, wherein up to 5% by weight, related to the total weight of the monomers of polymer 1 or polymer 2 of a Chain-transfer agent is added to the monomers.
19. Process according to item 17 or 18, wherein the Chain-transfer agent is ethylhexylthioglycolate or n-butylmercaptan, n-dodecylmercaptan or 2-mercaptoethanol or any mixtures thereof.
20. The use of a polymer mixture according to one or more items 1-12 for preparing the coating layer of dosage form according to any of items 13 to 16 with a sustained release profile and resistance against the influence of ethanol.

### EXAMPLES

### Abbreviations

TGEH=2-ethylhexyl thioglycolat
EHMA=ethylhexyl methacrylate
EMA=ethyl methacrylate
HEMA=hydroxyethyl methacrylate
TMAEMC=trimethylammonium-ethyl-methacrylate-chloride
MMA=methyl methacrylate
SDS=sodium dodecyl sulfate
TEC=triethyl citrate
NM=EUDRAGIT® NM, a copolymer comprising polymerized units from 70% by weight ethyl acrylate and 30% by weight methyl methacrylate
RL=EUDRAGIT® RL, a copolymer comprising polymerized units of from 60% by weight of methyl methacrylate, 30% by weight of ethyl acrylate and 10% by weight of 2-trimethylammoniumethyl methacrylate chloride.
RS=EUDRAGIT® RS, a copolymer comprising polymerized units from 65% by weight of methyl methacrylate, 30% by weight of ethyl acrylate and 5% by weight of 2-trimethylammoniumethyl methacrylate chloride.

Measurement Methods

The measurement of the percentage amount of active ingredient released can be carried out, for example, by on-line UV spectroscopy at a wavelength suitable for the respective active compound. HPLC determination is also possible. The methodology is familiar to a person skilled in the art.

The release of active ingredient can be determined according to USP, in particular USP 32-NF27, General Chapter <711>, Dissolution, Apparatus 2 (basket), Method <724>“Delayed Release (Enteric Coated) Articles—General, General Drug Release Standard”, Method B (100 rpm, 37° C.), type I basket, with the following modification: The pharmaceutical forms are tested at pH 1.2 for the first 2 hours using 0.1 N HCl medium or at pH 6.8 using a phosphate buffer (European Pharmacopoeia (EP)), which corresponds to an artificial intestinal medium.

The measurement in the ethanol containing aqueous pH 1.2 medium is carried out using 40% ethanol (w/w) in the medium. If appropriate or required for a certain controlled release pharmaceutical composition, depending on the active ingredient included and the type and size of the release of form (small or large pellet or small or large tablet) instead of the basket method the paddle method may be used with 50, 100 or 150 rpm.

In vitro drug release of coated pellets from Example 2 was tested in triplicates using USP I (basket) apparatus. Measurement was carried out at 150 RPM in 900 mL dissolution vessels. Dissolution was tested in 0.1 N HCl (pH 1.2) with and without 40% (w/w) EtOH for 2 h. Subsequently, medium was fully replaced by pH 6.8 EP buffer and drug release was monitored for another 8 h. API concentration was quantified via UVNIS spectroscopy. Results are presented as mean average±standard deviation, relatively to the total drug concentration in the respective vessel after homogenization.

The MFFT was determined according to the Standard of the International Organisation for Standardization DIN ISO 2115 with the exception of point 6.1 in that the maximum difference of the most distant metering points was set to 50° C.

Gel permeation chromatography (GPC) was used to determine the number- and weight-average molecular weights (Mₙ, M_{w}) and the polydispersity (D) of the inventive polymers in the examples according to DIN 55672-1. Equipment consisted of four PSS SDV columns (Mainz, Germany) plus pre-column of the same type, a column oven operating at 35° C., an Agilent (Series 1100, Santa Clara, USA) pump plus RI-detector of the same series. A 0.02 M solution of 2-(Diethylamino)ethylamine (DEAEA) in Tetrahydrofuran (THF) was used as eluent at a flow rate of 1 ml/min. Samples were dissolved in the eluent at concentrations of 2 mg/mL. For each measurement 100 μL polymer solution was injected. The values for Mn and Mw were calculated based on calibration curves generated by Poly(methyl methacrylate) standards.

EUDRAGIT® reference samples were measured using the eluent N,N-dimethylacetamide (DMAc). Method for EUDRAGIT® RL/RS is described in more detail by Adler M. et al. (e-Polymers, ISSN (Online) 1618-7229, ISSN (Print) 2197-4586, DOI: https://doi.org/10.1515/epoly.2005.5.1.602). Method for EUDRAGIT® NM is described in more detail by Adler M. et al. (e-Polymers, ISSN (Online) 1618-7229, ISSN (Print) 2197-4586, DOI: https://doi.org/10.1515/epoly.2004.4.1.608).

DSC measurement of the dry polymer substance was conducted according to DIN EN ISO 11357-2 with a heating rate of 20° C./min. The midpoint glass transition temperature T_{mg} was determined by half step height method as described in section 10.1.2 of DIN EN ISO 11357-2.

### Example 1: Emulsion Polymerization

Table 1 summarizes the compositions of polymers 1 and 2 (according to the invention) and commercially available polymers EUDRAGIrRL, EUDRAGIT® RS and EUDRAGIT® NM (comparative, not according to the invention) with sustained release characteristics.

Abbreviations in table 1: (%=% by weight, Da=Dalton, M_{w}=weight-average molecular weight, T_{mg}=midpoint glass transition temperature, MFFT=minimum film forming temperature, D=Dispersity Index)

Procedure is described exemplarily for Polymer 1 (see Table 1). Polymer 2 was manufactured in the same manner. Setup consisted of a 1 L reaction vessel equipped with lid, agitator, condenser, nitrogen inlet and thermal sensor. Heating was carried out by a thermostat controlled water bath. A dosage pump with silicone tubes was used to dose monomer emulsion into the reaction mixture. In a first step, 534.0 g of water and 6.6 g of sodium dodecyl sulfate (SDS 15, 15.0% (w/w) aqueous solution) were dosed into the reactor, purged with nitrogen and the mixture was then heated to 80° C. In parallel, in a separate flask, monomer emulsion was prepared by mixing 21.3 g of SDS 15, 0.8 g of chain transfer agent (2-Ethylhexylthioglycolat, TGEH), 187.0 g (66.8% (w/w)) EHMA, 67.8 g (22.4% (w/w)) EMA, and 30.2 g (10.8% (w/w)) TMAEMC with 76.0 g of water. Stable emulsion was formed by stirring for 20 min. As soon as reaction mixture reached target temperature (80° C.), 6.0 mL of APS initiator (ammonium persulfate, 10% (w/w) aqueous solution) were pipetted into the reactor, followed by feeding of previously prepared monomer-emulsion. Feeding was carried out stepwise using two different rates (10 min at 1.5 g/min, followed by 120 min at 3.0 mg/min). During dosing, reaction temperature was held constant between 80 and 82° C. After complete monomer addition, reaction mixture was stirred for 30 min at 80° C. and then allowed to cool down to room temperature. In total 28.0 g SDS 15 solution were used (4.2 g SDS, 1.5% (w/w) based on polymer weight). Theoretic solid content of the resulting polymer dispersion is 30% (w/w). Dispersion was finally filtered through a 250 um gauze. Filtrate and polymer coagulate in the reactor were collected and dried for gravimetric analysis. Experimental solid content of the final dispersion was 29.2% (w/w), coagulate was <0.1%.

The MFFT was determined according to the Standard of the International Organisation for Standardization DIN ISO 2115 with the exception of point 6.1 in that the maximum difference of the most distant metering points is set to 50° C.

DSC measurement of the dry polymer substance was conducted according to DIN EN ISO 11357-2 with a heating rate of 20° C./min. Midpoint glass transition temperature T_{mg} was determined by half step height method as described in section 10.1.2 of DIN EN ISO 11357-2.

TABLE 1
Co-monomer composition of polymers together
with analytical characterization parameters

| | Co-monomer composition | | | | | | | |
|---|---|---|---|---|---|---|---|---|
| | EHMA | EMA | HEMA | TMAEMC | MMA | EA | T_{mg} | MFFT |
| Compound | [%] | [%] | [%] | [%] | [%] | [%] | [° C.] | [° C.] |
| Polymer 1 | 66.8 | 22.4 | | 10.8 | | | 10.5 | |
| Polymer 2 | 67.4 | 22.6 | 10 | | | | 31 | 31 |
| EUDRAGIT ® RL | | | | 10 | 60 | 30 | 70 | 40 |
| EUDRAGIT ® RS | | | | 5 | 65 | 30 | 65 | 45 |
| EUDRAGIT ® NM | | | | | 30 | 70 | 9 | 5 |

### Example 2: Coating of Polymer Dispersion on Metoprolol Succinate Pellets

Metoplolol Succinate pellets were obtained from Lee Pharma Limited (Telangana, India). Drug content is 40%. Particle size (determined by sieve analysis) is specified as follows: not more than 10% retains on sieve #16 ASTM and not more than 10% passes through sieve #25 ASTM (this relates to ca. 1 mm mean pellet diameter).

Dispersions of Polymer 1 and Polymer 2 (30% (w/w) polymer solid content) from Example 1 were used as is, or mixed in ratio 1:1.36 g talc (100% (w/w) compared to dry polymer mass) were suspended in 204 g water and homogenized using ultra turrax for 15 minutes. Subsequently, the prepared suspension was mixed with 120 g polymer dispersion (or dispersion mixture) and stirred for one hour. The amount of water for suspension of talc was calculated to result in a final spraying suspension of 20% (w/w) solid content. Coating experiments were carried out on a Huttlin (Schopfheim, Germany) Mycrolab (H00263), equipped with an ISMATEC (Wertheim, Germany) MCP flexible-tube pump (silicone tube, internal diameter 2 mm) and an inlet-air dehumidifier. A 0.8 mm spray nozzle was used. Atomizing air-pressure and micro-climate air-pressure were set to 0.6 and 0.4 bar, respectively. Product-bed temperature was held constant at ˜30° C., air-flow rate at ˜20 m³/h, spray rate between 10 and 15 g/min/kg. Process was stopped at desired polymer weight gain. Curing was performed in open trays for 24 h at 60° C.

EUDRAGIT® NM, EUDRAGIT® RL and EUDRAGIT® RS were used for comparison and film-coatings were prepared in the same manner. In case of EUDRAGIT® RUEUDRAGIT® RS, however, a plasticizer is required to enable film formation at applied spraying conditions. Therefore, Triethyl citrate (TEC, 20% (w/w) compared to dry polymer mass) was added to the EUDRAGIT® RUEUDRAGIT® RS-talc mixture and stirred for one hour prior to spraying process. Curing of all EUDRAGIT® coated dosage forms was performed (according to Evonik standard recommendations/Application Guidelines 12th ed.) in open trays at 40° C. for 24 hours.

### Example 3: Dissolution Testing in Pure Media and Hydroalcoholic Media

In vitro drug release of coated pellets from Example 2 was tested in triplicates using USP I (basket) apparatus. Measurement was carried out at 150 RPM in 900 mL dissolution vessels. Dissolution was tested in 0.1 N HCl (pH 1.2) with and without 40% (w/w) ethanol (EtOH) for 2 h. Subsequently, medium was fully replaced by pH 6.8 EP buffer (without ethanol) and drug release was monitored for another 8 h. API concentration was quantified via UVNIS spectroscopy. Results are presented as mean average±standard deviation, relatively to the total drug concentration in the respective vessel after homogenization.

TABLE 2
Dissolution tests with and without ethanol of different polymers coated onto Metoprolol succinate pellets

| | Polymer | | | | | | | | | |
|---|---|---|---|---|---|---|---|---|---|---|
| | | | | | Mixture | | | | | |
| | | | | | Polymer ½ | | EUDRAGIT ® | | EUDRAGIT ® | |
| | Polymer 1 | | Polymer 2 | | (ratio 1:1) | | NM | | RS/RL (9:1) | |
| Talcum [% (w/w) compared to dry polymer | 100 | | 100 | | 100 | | 100 | | 50 | |
| mass] | | | | | | | | | | |
| TEC [% (w/w) compared to dry polymer | 0 | | 0 | | 0 | | 0 | | 20 | |
| mass] | | | | | | | | | | |
| Polymer weight gain [% (w/w) compared to | 10 | | 10 | | 10 | | 6 | | 15 | |
| drug substrate mass] | | | | | | | | | | |
| Active release without/with 40% EtOH | | | | | | | | | | |
| (w/w) | | | | | | | | | | |
| 5 min (pH 1.2 without/with EtOH) | 0 | 0 | −0.21 | 0.06 | 0.32 | 0.39 | 0.03 | 0.44 | 2.04 | 71.06 |
| 30 min (pH 1.2 without/with EtOH) | 2.08 | 0.79 | −0.05 | 0.24 | 20.22 | 20.72 | 1.03 | 7.51 | 4.33 | 102.18 |
| 1 h (pH 1.2 without/with EtOH) | 11.66 | 3.04 | −0.04 | 1.16 | 41.21 | 47.29 | 4.18 | 23.76 | 5.29 | 100.84 |
| 1.5 h (pH 1.2 without/with EtOH) | 27.09 | 7.01 | −0.05 | 5.75 | 57.40 | 62.51 | 8.57 | 38.88 | 5.90 | 100.30 |
| 2 h (pH 1.2 without/with EtOH) | 43.26 | 12.76 | −0.03 | 22.53 | 67.95 | 71.56 | 13.92 | 67.88 | 11.04 | 99.71 |
| 2.5 h (pH 6.8 without EtOH) | 55.26 | 13.07 | 0.05 | 30.28 | 75.17 | 75.73 | 19.28 | 78.26 | 25.56 | 99.79 |
| 3 h (pH 6.8 without EtOH) | 63.45 | 16.42 | −0.01 | 37.75 | 79.81 | 80.34 | 24.52 | 81.25 | 36.87 | 99.81 |
| 3.5 h (pH 6.8 without EtOH) | 69.92 | 20.42 | 0.03 | 45.55 | 83.54 | 84.26 | 27.77 | 82.88 | 41.59 | 99.81 |
| 4 h (pH 6.8 without EtOH) | 75.13 | 24.74 | 0.08 | 52.77 | 86.71 | 87.42 | 40.64 | 87.97 | 45.87 | 99.82 |
| 5 h (pH 6.8 without EtOH) | 82.75 | 33.84 | 0.48 | 64.78 | 91.47 | 92.07 | 51.72 | 91.09 | 49.54 | 99.82 |
| 6 h (pH 6.8 without EtOH) | 87.88 | 42.74 | 1.50 | 73.99 | 94.63 | 95.01 | 60.61 | 93.47 | 52.83 | 99.85 |
| 7 h (pH 6.8 without EtOH) | 91.37 | 50.84 | 4.72 | 80.91 | 96.59 | 96.80 | 67.68 | 95.10 | 55.63 | 99.84 |
| 8 h (pH 6.8 without EtOH) | 93.87 | 57.94 | 12.18 | 86.20 | 97.78 | 97.94 | 73.39 | 96.16 | 60.27 | 99.82 |
| 9 h (pH 6.8 without EtOH) | 95.60 | 63.86 | 24.08 | 90.22 | 98.50 | 98.58 | 77.98 | 96.94 | 63.67 | 99.85 |
| 10 h (pH 6.8 without EtOH) | 96.74 | 69.07 | 37.40 | 93.25 | 98.98 | 99.02 | 81.77 | 97.45 | 66.33 | 99.88 |

| | | | | | |
|---|---|---|---|---|---|
| Arithmetic average (bold figures) | −35.47 | 65.34 | 2.73 | 41.35 | 53.53 |
| n for calculation | 7 | 3 | 6 | 10 | 11 |
| Ethanol resistance | no | no | yes | no | no |

1: A polymer mixture, comprising:
10 to 90% by weight of a polymer 1, and
10 to 90% by weight a polymer 2,
wherein the weight percentages of polymer 1 and polymer 2 add up to 100%,
wherein polymer 1 is a polymer, polymerized from a monomer mixture comprising monomers
(a1) 70 to 95% by weight of a C₁-C₁₂ alkylester of acrylic acid or of methacrylic acid, and
(b1) 5 to 30% by weight of a C₂-C₈ alkyl ester of acrylic acid or of methacrylic acid with a quaternary cationic group in the alkyl group,
wherein polymer 2 is polymerized from a monomer mixture comprising monomers
(a2) 70 to 95% by weight of a C₁-C₁₂ alkylester of acrylic acid or of methacrylic acid, and
(c2) 5 to 30% by weight of a C₂-C₆ hydroxy-alkylester of acrylic acid or methacrylic acid,
wherein in polymer 1, the monomers (a1) and (hl) add up to from 98 to 100% by weight, and
wherein in polymer 2, independent from polymer 1, the monomers (a2) and (c2) add up to from 98 to 100% by weight.

2: The polymer mixture as claimed in claim 1, wherein the C₁-C₁₂ alkyl ester of acrylic acid or of methacrylic acid (a1) or (a2) is 2-ethylhexyl methacrylate, ethyl or methacrylate, or a mixture thereof.

3: The polymer mixture as claimed in claim 2, wherein 2-ethylhexyl methacrylate and ethyl methacrylate are comprised in (a1) or in (a2) or in both at a ratio by weight from 5:1 to 1:1.

4: The polymer mixture as claimed in claim 1, wherein the C₂-C₈ alkyl ester of acrylic acid or of methacrylic acid with a quaternary cationic group in the alkyl group (b1) is 2-trimethylammonium-ethyl-methacrylate-chloride.

5: The polymer mixture as claimed in claim 1, wherein the C₂-C₆ hydroxy-alkylester of acrylic acid or of methacrylic acid (c2) is selected from the group consisting of 2 hydroxyethyl methacrylate, 2-hydroxypropyl methacrylate, 3-hydroxypropyl methacrylate, 2,3-dihydroxypropyl methacrylate, 2-hydroxyethyl acrylate, 2-hydroxypropyl acrylate, 3-hydroxypropyl acrylate, 2,3-dihydroxypropyl acrylate, and mixtures thereof.

6: The polymer mixture according to claim 1, wherein polymers 1 and 2, independent from each other, comprise 0 to 2% by weight of further vinylically copolyrnerizable monomers.

7: The polymer mixture according to claim 1, wherein a minimum film forming temperature of polymer 1 or polymer 2 or both is 35° C. or lower.

8: The polymer mixture according to claim 1, wherein a midpoint glass transition temperature of polymer 1 is in a range from −10 to 30.

9: The polymer mixture according to claim 1, wherein a midpoint glass transition temperature of polymer 2 is in a range from 0 to 50° C.

10: The polymer mixture according to claim 1, comprising a ratio of polymer 1 to polymer 2 of from 5:1 to 1:5.

11: The polymer mixture according to claim 1, wherein a weight average molecular weight Mw of polymer 1 or polymer 2 is from 50,000 to 200,000 Dalton.

12: A dosage form, comprising:
a core, comprising
a biologically active ingredient, and
a coating layer coated onto the core,
wherein the coating layer is core,
the polymer mixture as claimed in claim 1, and
optionally, a pharmaceutical or nutraceutical excipient.

13: A process for preparing the polymer mixture as claimed in claim 1, comprising:
polymerizing polymer 1 and polymer 2 independently from each other from their monomers in the presence of a polymerization-initiator and optionally a chain-transfer agent,
wherein the polymerization is by bulk polymerization, suspension polymerization, or emulsion polymerization, and
subsequently mixing polymer 1 and polymer 2.

14: A method for preparing a coating layer of a dosage form, the method comprising:
applying the polymer mixture according to claim 1 to a core,
wherein the dosage form has a sustained release profile and resistance against the influence of ethanol.

15: The polymer mixture according to claim 1, wherein in polymer 1, the monomers (a1) and (b1) add up to 100%, and wherein in polymer 2, independent from polymer 1, the monomers (a2) and (c2) add up to 100%.

16: The polymer mixture according to claim 3, wherein 2-ethylhexyl methacrylate and ethyl methacrylate are comprised in (a1) or in (a2) or in both at a ratio by weight from 4:1 to 1:1.

17: The polymer mixture according to claim 6, wherein polymer 1 or 2 or both, independent from each other, do not comprise any vinylically copolymerizable monomers.

18: The polymer mixture according to claim 7, wherein the minimum film forming temperature of polymer 1 or polymer 2 or both is 10 to 20° C.

19: The polymer mixture according to claim 8, wherein the midpoint glass transition temperature of polymer 1 is in the range from 0 to 20° C.

20: The polymer mixture according to claim 9, wherein the midpoint glass transition temperature of polymer 2 is in the range from 20 to 45° C.
